(19) United States
(12) Patent Application Publication
(10) Pub. No.: US 2002/0090646 A1
(43) Pub. Date: Jul. 11, 2002

(54) Calcitonin-related molecules

(75) Inventors: Chuan-Fa Liu, Longmont, CO (US); William S. Marshall, Boulder, CO (US); Angela Reynolds, Evergreen, CO (US)

Correspondence Address: U.S. Patent Operations/ TJG, Dept. 4300, M/S 27-4-A, AMGEN INC., One Amgen Center Drive, Thousand Oaks, CA, 91320-1799, US

(73) Assignee: Amgen Inc.

(21) Appl. No.: 09/847,712

(22) Filed: May 2, 2001

Related U.S. Application Data
(60) Provisional application No. 60/201,511, filed on May 3, 2000.

(51) Int. Cl. G01N 33/53; C07K 16/46

(52) U.S. Cl. 435/7.1; 530/389.1

(57) ABSTRACT

The present invention concerns therapeutic agents that modulate the activity of CT receptor. In accordance with the present invention, modulators of CT receptor comprise:

a. a CT receptor modulating domain, preferably the amino acid sequence of SEQ ID NO: 7, or sequences derived therefrom by phage display, RNA-peptide screening, or the other techniques; and

b. a vehicle, such as a polymer (e.g., PEG or dextran) or an Fc domain, which is preferred;

wherein the vehicle is covalently attached to the CT receptor modulating domain. The vehicle and the CT receptor modulating domain may be linked through the N- or C-terminus of the CT receptor modulating domain, as described further below. The preferred vehicle is an Fc domain, and the preferred Fc domain is an IgG Fc domain. Preferred CT receptor modulating domains comprise the amino acid sequences described in Table 1. Other CT receptor modulating domains can be generated by phage display, RNA-peptide screening and the other techniques mentioned herein.

Further in accordance with the present invention is a process for making CT receptor modulators, which comprises:

a. selecting at least one peptide that binds to the CT receptor; and

b. covalently linking said peptide to a vehicle.

The preferred vehicle is an Fc domain. Step (a) is preferably carried out by selection from the peptide sequences in Table 1 hereinafter or from phage display, RNA-peptide screening, or the other techniques mentioned herein.

This application claims the benefit of U.S. Provisional Application No. 60/201,511, filed May 3, 2000, which is hereby incorporated by reference.

### BACKGROUND OF THE INVENTION

Calcitonin (CT) is a peptide hormone with 32 amino acid residues and has a C-terminal proline-amide and a 7-residue disulfide loop at the N-terminus. CT acts through inhibiting the bone-resorbing activity of osteoclasts. The C-terminal amide is essential for bioactivity, while the disulfide bridge is not so important. Although with only 50% homology to human CT, salmon CT (sCT) exhibits greater affinity to human receptors and is widely used for therapeutic applications. The administration of pharmacological doses of sCT has dramatic effect on the development of bone lesions, can prevent osteomalacia and lower bone resorption. Synthetic sCT is an important agent for the treatment of postmenopausal osteoporosis. Paget's disease and other forms of osteodystrophia are also successfully treated with sCT. Very importantly, CT is extremely effective in the treatment of bone pain, which makes it distinctive from other therapeutic agents for osteoporosis. Currently, synthetic salmon CT has an annual worldwide revenue of about $400 million.

To date, no recombinant or modified proteins employing peptide modulators of the CT receptor have been disclosed. Recombinant and modified proteins are an emerging class of therapeutic agents. Useful modifications of protein therapeutic agents include combination with the “Fc” domain of an antibody and linkage to polymers such as polyethylene glycol (PEG) and dextran. Such modifications are discussed in detail in a patent application entitled, “Modified Peptides as Therapeutic Agents,” U.S. Ser. No. 09/428,082, PCT appl. no. WO 99/25044, which is hereby incorporated by reference in its entirety.

A much different approach to development of therapeutic agents is peptide library screening. The interaction of a protein ligand with its receptor often takes place at a relatively large interface. However, as demonstrated for human growth hormone and its receptor, only a few key residues at the interface contribute to most of the binding energy. Clackson et al. (1995), Science 267: 383-6. The bulk of the protein ligand merely displays the binding epitopes in the right topology or serves functions unrelated to binding. Thus, molecules of only “peptide” length (2 to 90 amino acids) can bind to the receptor protein of a given large protein ligand. Such peptides may mimic the bioactivity of the large protein ligand (“peptide agonists”) or, through competitive binding, inhibit the bioactivity of the large protein ligand (“peptide antagonists”).

Phage display peptide libraries have emerged as a powerful method in identifying such peptide agonists and antagonists. See, for example, Scott et al. (1990), Science 249: 386; Devlin et al. (1990), Science 249: 404; U.S. Pat. No. 5,223,409, issued Jun. 29, 1993; U.S. Pat. No. 5,733,731, issued Mar. 31, 1998; U.S. Pat. No. 5,498,530, issued Mar. 12, 1996; U.S. Pat. No. 5,432,018, issued Jul. 11, 1995; U.S. Pat. No. 5,338,665, issued Aug. 16, 1994; U.S. Pat. No. 5,922,545, issued Jul. 13, 1999; WO 96/40987, published Dec. 19, 1996; and WO 98/15833, published Apr. 16, 1998 (each of which is incorporated by reference in its entirety). In such libraries, random peptide sequences are displayed by fusion with coat proteins of filamentous phage. Typically, the displayed peptides are affinity-eluted against an antibody-immobilized extracellular domain of a receptor. The retained phages may be enriched by successive rounds of affinity purification and repropagation. The best binding peptides may be sequenced to identify key residues within one or more structurally related A-684 families of peptides. See, e.g., Cwirla et al. (1997), Science 276: 1696-9, in which two distinct families were identified. The peptide sequences may also suggest which residues may be safely replaced by alanine scanning or by mutagenesis at the DNA level. Mutagenesis libraries may be created and screened to further optimize the sequence of the best binders. Lowman (1997), Ann. Rev. Biophys. Biomol. Struct. 26: 401-24.

Another biological approach to screening soluble peptide mixtures uses yeast for expression and secretion (Smith et al. (1993), Mol. Pharmacol. 43: 741-8) to search for peptides with favorable therapeutic properties. Hereinafter, this and related methods are referred to as “yeast-based screening.”

Still other methods compete with phage display in peptide research. A peptide library can be fused to the carboxyl terminus of the lac repressor and expressed in E. coli. Another E. coli-based method allows display on the cell's outer membrane by fusion with a peptidoglycan-associated lipoprotein (PAL). Hereinafter, these and related methods are collectively referred to as “E. coli display.” In another method, translation of random RNA is halted prior to ribosome release, resulting in a library of polypeptides with their associated RNA still attached. Hereinafter, this and related methods are collectively referred to as “ribosome display.” Other methods employ peptides linked to RNA; for example, PROfusion technology, Phylos, Inc. See, for example, Roberts & Szostak (1997), Proc. Natl. Acad. Sci. USA, 94: 12297-303. Hereinafter, this and related methods are collectively referred to as “RNA-peptide screening.” Chemically derived peptide libraries have been developed in which peptides are immobilized on stable, non-biological materials, such as polyethylene rods or solvent-permeable resins. Another chemically derived peptide library uses photolithography to scan peptides immobilized on glass slides. Hereinafter, these and related methods are collectively referred to as “chemical-peptide screening.” Chemical-peptide screening may be advantageous in that it allows use of D-amino acids and other unnatural analogues, as well as non-peptide elements. Both biological and chemical methods are reviewed in Wells & Lowman (1992), Curr. Opin. Biotechnol. 3: 355-62.

In the case of known bioactive peptides, rational design of peptide ligands with favorable therapeutic properties can be completed. In such an approach, one makes stepwise changes to a peptide sequence and determines the effect of the substitution upon bioactivity or a predictive biophysical property of the peptide (e.g., solution structure). Hereinafter, these techniques are collectively referred to as “rational design.” In one such technique, one makes a series of peptides in which one replaces a single residue at a time with alanine. This technique is commonly referred to as an “alanine walk” or an “alanine scan.” When two residues (contiguous or spaced apart) are replaced, it is referred to as a “double alanine walk.” The resultant amino acid substitutions can be used alone or in combination to result in a new peptide entity with favorable therapeutic properties.

Structural analysis of protein-protein interaction may also be used to suggest peptides that mimic the binding activity of large protein ligands. In such an analysis, the crystal structure may suggest the identity and relative orientation of critical residues of the large protein ligand, from which a peptide may be designed. See, e.g., Takasaki et al. (1997), Nature Biotech. 15: 1266-70. Hereinafter, these and related methods are referred to as “protein structural analysis.” These analytical methods may also be used to investigate the interaction between a receptor protein and peptides selected by phage display, which may suggest further modification of the peptides to increase binding affinity.

Conceptually, one may discover peptide mimetics of any protein using phage display, RNA-peptide screening, and the other methods mentioned above.

### SUMMARY OF THE INVENTION

The present invention concerns therapeutic agents that modulate the activity of CT receptor. In accordance with the present invention, modulators of CT receptor comprise:

a) a CT receptor modulating domain, preferably the amino acid sequence of human or salmon calcitonin, or sequences derived by phage display, RNA-peptide screening, or the other techniques mentioned above; and

b) a vehicle, such as a polymer (e.g., PEG or dextran) or an Fc domain, which is preferred;

wherein the vehicle is covalently attached to the CT receptor modulating domain. The vehicle and the CT receptor modulating domain may be linked through the N- or C-terminus of the CT receptor modulating domain, as described further below. The preferred vehicle is an Fc domain, and the preferred Fc domain is an IgG Fc domain. Preferred CT receptor modulating domains comprise the amino acid sequences described hereinafter in Table 1. Additional CT receptor modulating domains within the scope of this invention are described in WO 99/31131, published 24 June 1999, which is hereby incorporated by reference in its entirety. Other CT receptor modulating domains can be generated by phage display, RNA-peptide screening and the other techniques mentioned herein.

Further in accordance with the present invention is a process for making CT receptor modulators, which comprises:

a. selecting at least one peptide that binds to CT receptor; and

b. covalently linking said peptide to a vehicle.

The preferred vehicle is an Fc domain. Step (a) is preferably carried out by selection from the peptide sequences in Table 1 hereinafter or from phage display, RNA-peptide screening, or the other techniques mentioned herein.

The compounds of this invention may be prepared by standard synthetic methods, recombinant DNA techniques, or any other methods of preparing peptides and fusion proteins. Compounds of this invention that encompass non-peptide portions may be synthesized by standard organic chemistry reactions, in addition to standard peptide chemistry reactions when applicable.

The primary use contemplated for the compounds of this invention is as therapeutic or prophylactic agents. The vehicle-linked peptide may have activity comparable to—or even greater than—the natural ligand mimicked by the peptide.

The compounds of this invention may be used for therapeutic or prophylactic purposes by formulating them with appropriate pharmaceutical carrier materials and administering an effective amount to a patient, such as a human (or other mammal) in need thereof. Other related aspects are also included in the instant invention.

Numerous additional aspects and advantages of the present invention will become apparent upon consideration of the figures and detailed description of the invention.

### BRIEF DESCRIPTION OF THE FIGURES

FIG. 1 shows exemplary Fc dimers that may be derived from an IgG1 antibody. “Fc” in the figure represents any of the Fc variants within the meaning of “Fc domain” herein. “X¹” and “X²” represent peptides or linker-peptide combinations as defined hereinafter. The specific dimers are as follows:

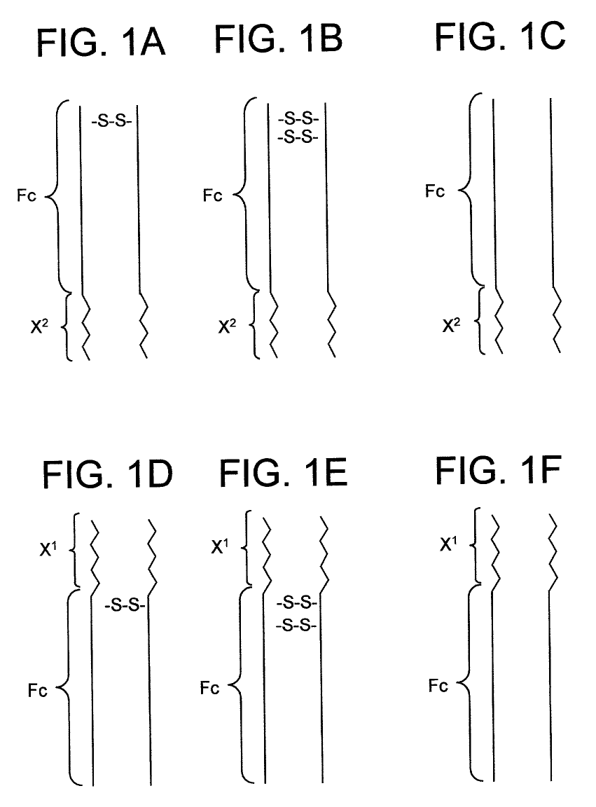

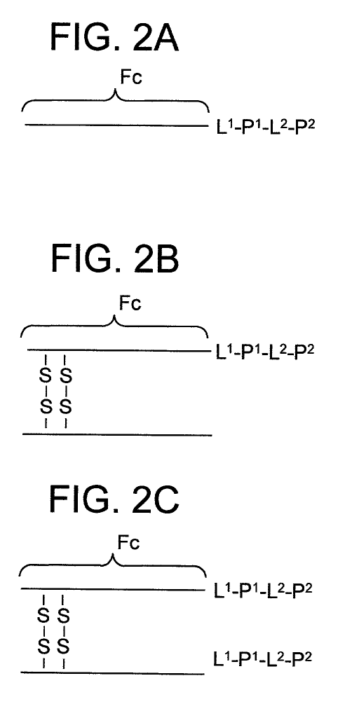

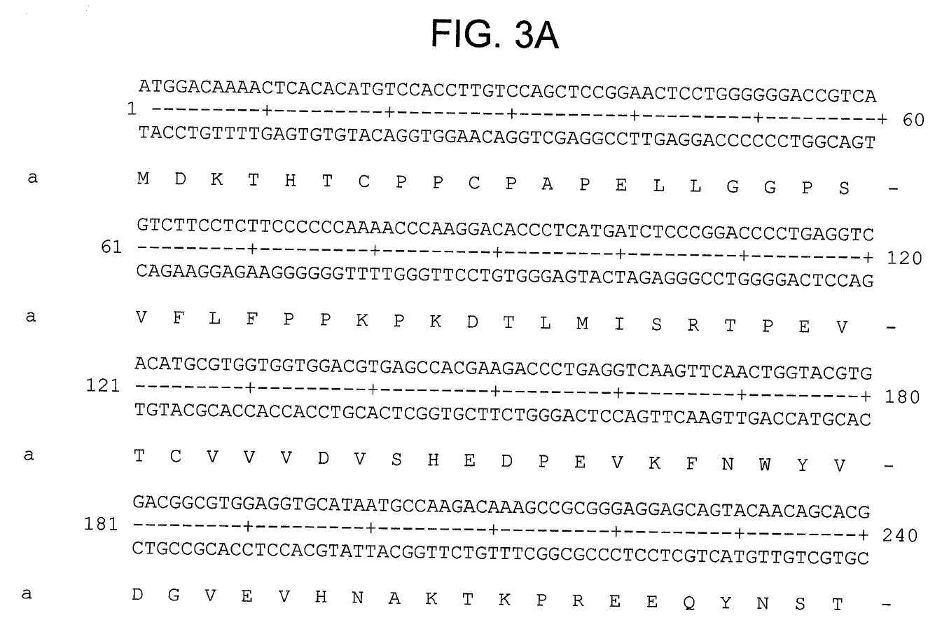

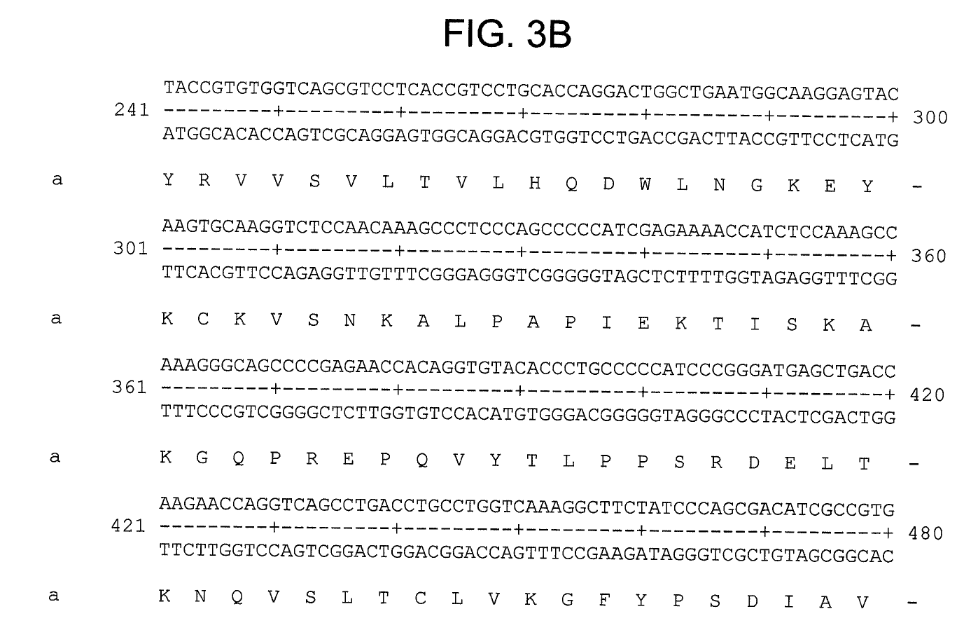

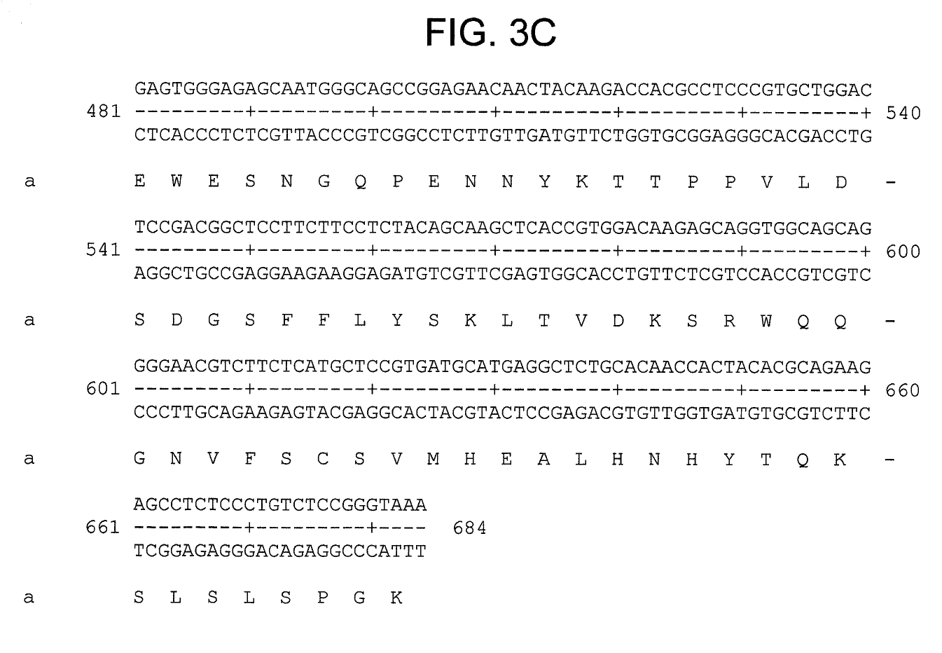

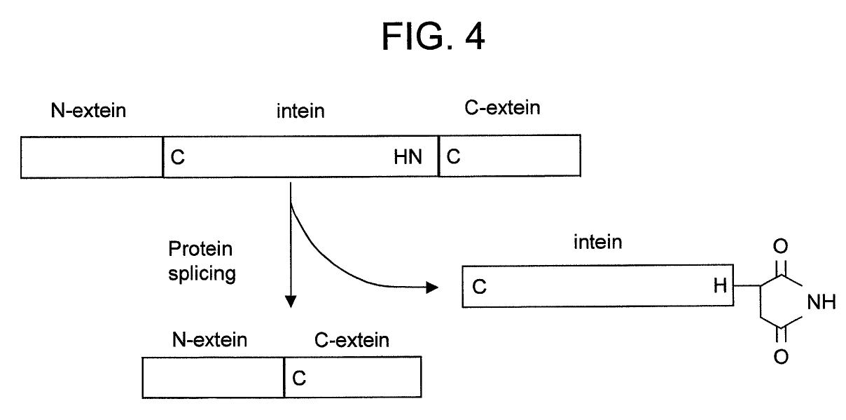

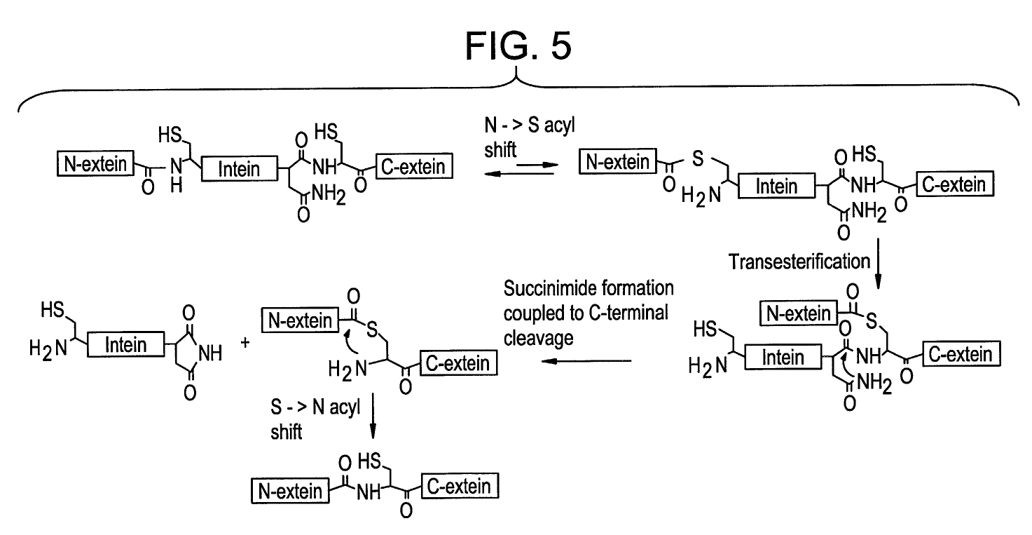

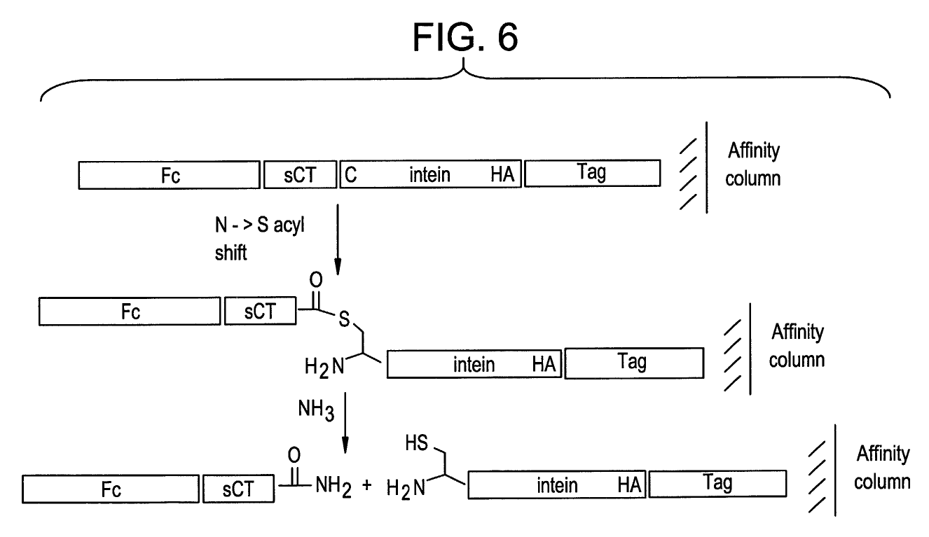

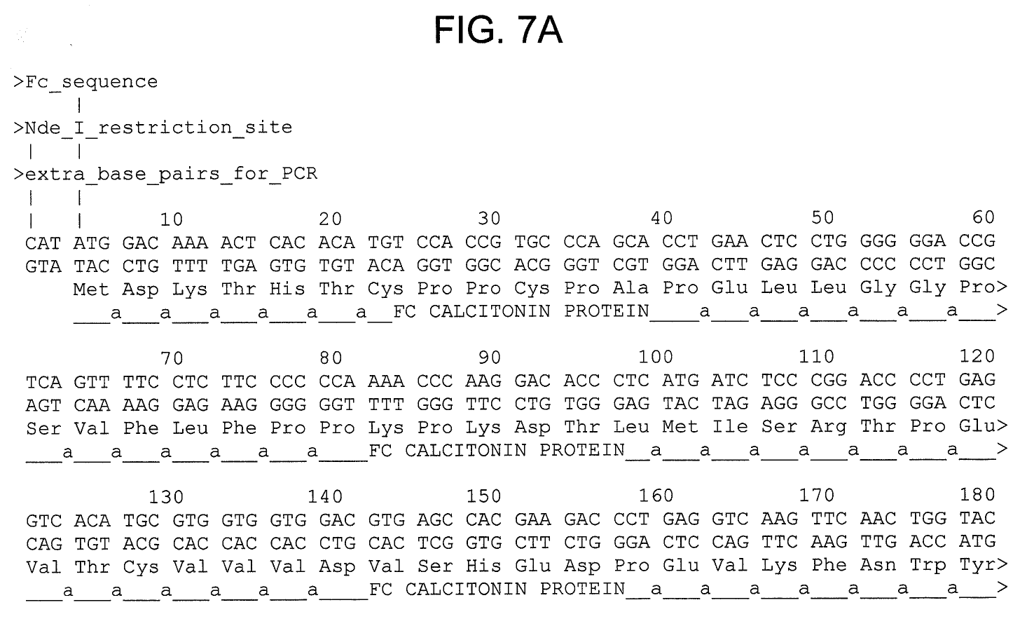

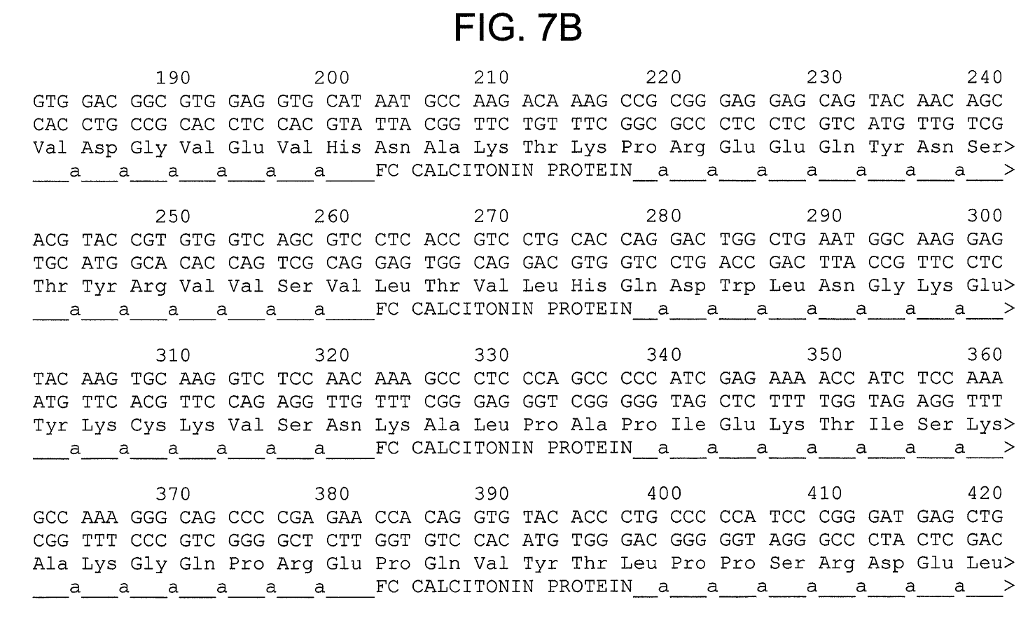

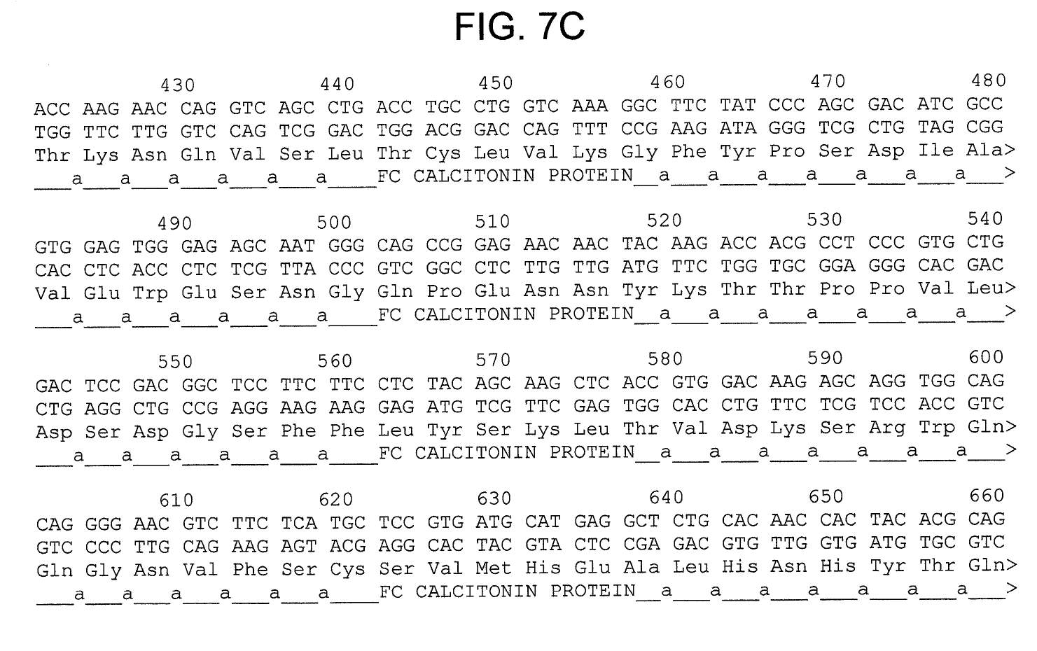

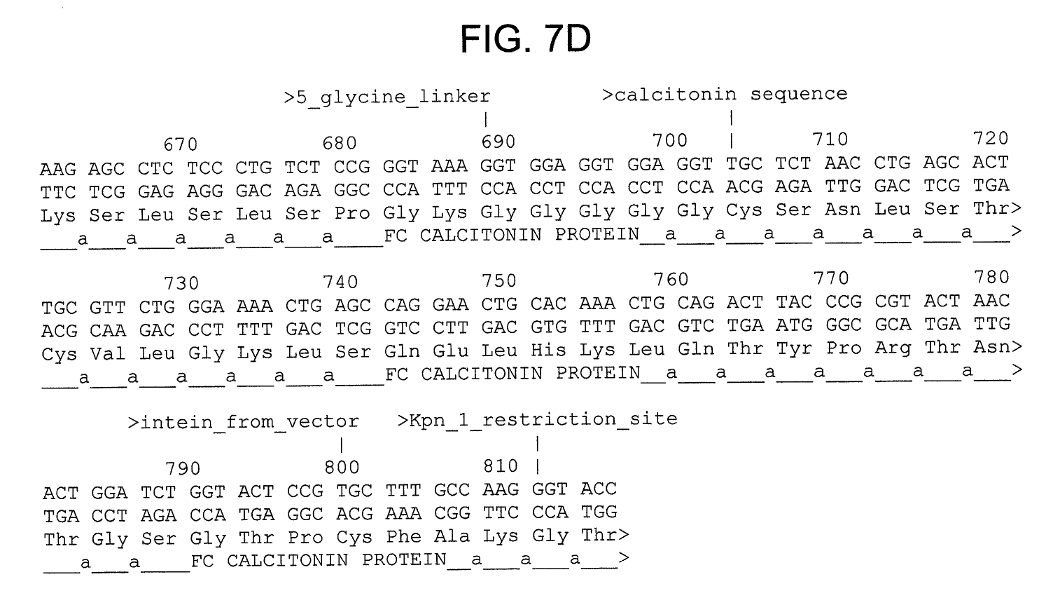

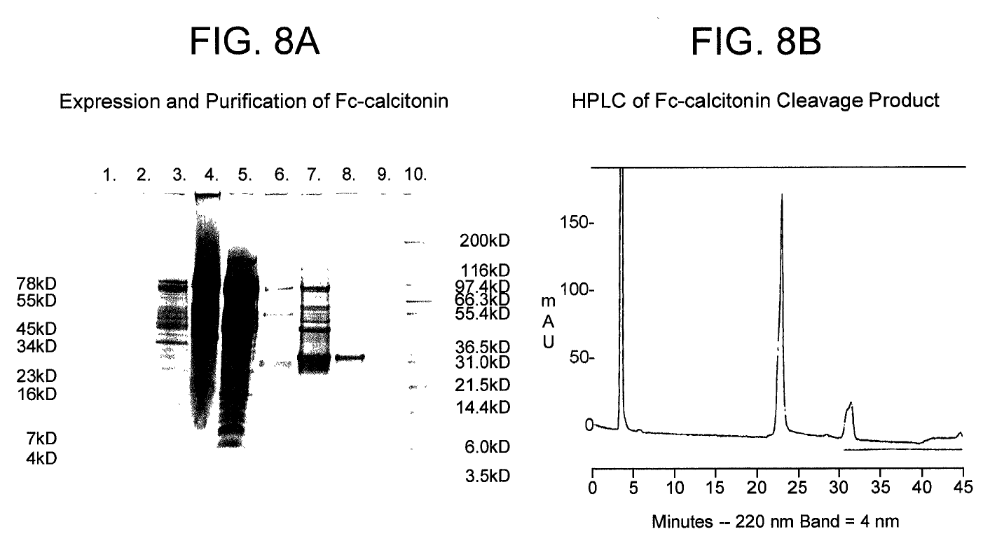

A, D: Single disulfide-bonded dimers. IgG1 antibodies typically have two disulfide bonds at the hinge region between the constant and variable domains. The Fc domain in FIGS. 2A and 2D may be formed by truncation between the two disulfide bond sites or by substitution of a cysteinyl residue with an unreactive residue (e.g., alanyl). In FIG. 2A, the Fc domain is linked at the amino terminus of the peptides; in 2D, at the carboxyl terminus.

B, E: Doubly disulfide-bonded dimers. This Fc domain may be formed by truncation of the parent antibody to retain both cysteinyl residues in the Fc domain chains or by expression from a construct including a sequence encoding such an Fc domain. In FIG. 2B, the Fc domain is linked at the amino terminus of the peptides; in 2E, at the carboxyl terminus.

C, F: Noncovalent dimers. This Fc domain may be formed by elimination of the cysteinyl residues by either truncation or substitution. One may desire to eliminate the cysteinyl residues to avoid impurities formed by reaction of the cysteinyl residue with cysteinyl residues of other proteins present in the host cell. The noncovalent bonding of the Fc domains is sufficient to hold together the dimer. Other dimers may be formed by using Fc domains derived from different types of antibodies (e.g., IgG2, IgM).

FIG. 2 shows the structure of preferred compounds of the invention that feature tandem repeats of the pharmacologically active peptide. FIG. 2A shows a single chain molecule and may also represent the DNA construct for the molecule. FIG. 2B shows a dimer in which the linker-peptide portion is present on only one chain of the dimer. FIG. 2C shows a dimer having the peptide portion on both chains. The dimer of FIG. 2C will form spontaneously in certain host cells upon expression of a DNA construct encoding the single chain shown in FIG. 3A. In other host cells, the cells could be placed in conditions favoring formation of dimers or the dimers can be formed in vitro.

FIG. 3 shows exemplary nucleic acid and amino acid sequences (SEQ ID NOS: 1 and 2, respectively) of human IgG1 Fc that may be used in this invention.

FIG. 4 shows a general scheme of protein splicing that is useful in preparing the fusion molecules of the present invention. This scheme is further described in Example 1 hereinafter.

FIG. 5 shows a mechanism of protein splicing that is useful in preparing the fusion molecules of the present invention. This mechanism is further described in Example 1 hereinafter.

FIG. 6 shows a proposed process for preparation of the fusion molecules of the present invention having a C-terminal amide. This process is further described in Example 1 hereinafter.

FIGS. 7A and 7B show the sequence of an Fc-calcitonin fusion molecule. The sequence segments corresponding to the Fc, linker, and calcitonin sequences are identified. Restriction sites and other features are also identified.

FIG. 8A shows expression and purification of the Fc-calcitonin fusion gene using the IMPACT-CN system. Lane 1: Protein Marker, SeeBlue (Ambion), Lane 2: Crude extracts from uninduced cells, Lane 3:

Crude extracts from cells induced at 15 C for 5 hours., Lane 4: Cell pellet, Lane 5: Clarified crude extract from induced cells., Lane 6: SDS stripping of proteins bound to the chitin column., Lane 7: Eluted Fc-calcitonin in 25 NH3HCO3, without MESA 48 hour incubation, Lane 8: Eluted Fc-calcitonin in NH3HCO3 and MESA overnight incubation, Lane 9: Eluted Fc-calcitonin in NH3HCO3 withoust MESA overnight incubation, Lane 10:

Protein Marker, Mark 12 (Ambion).

FIG. 8B shows the HPLC profile of Fc-Calcitonin. The 32kD Fc-calcitonin was analyzed on a RP-HPLC using a Vydac C4, 10 u 4.6×250 mm column. Gradient=5%-75%B in 35 min; A=0.1%TFA/H2O, B=80% ACN/H2O (0.05% TFA); flow rate=1 mL/min, lanbda=220 mm.

### DETAILED DESCRIPTION OF THE INVENTION

Definition of Terms

The terms used throughout this specification are defined as follows, unless otherwise limited in specific instances.

The term “comprising” means that a compound may include additional amino acids on either or both of the N- or C-termini of the given sequence. Of course, these additional amino acids should not significantly interfere with the activity of the compound.

The term “acidic residue” refers to amino acid residues in D- or L-form having sidechains comprising acidic groups. Exemplary acidic residues include D and E.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary neutral amino acid residues include M, G, A, V, I, L and norleucine (Nle).

The term “vehicle” refers to a molecule that prevents degradation and/or increases half-life, reduces toxicity, reduces immunogenicity, or increases biological activity of a therapeutic protein. Exemplary vehicles include an Fc domain (which is preferred) as well as a linear polymer (e.g., polyethylene glycol (PEG), polylysine, dextran, etc.); a branched-chain polymer (see, for example, U.S. Pat. No. 4,289,872 to Denkenwalter et al., issued Sep. 15, 1981; 5,229,490 to Tam, issued Jul. 20, 1993; WO 93/21259 by Frechet et al., published Oct. 28, 1993); a lipid; a cholesterol group (such as a steroid); a carbohydrate or oligosaccharide (e.g., dextran); or any natural or synthetic protein, polypeptide or peptide that binds to a salvage receptor. Vehicles are further described hereinafter.

The term “native Fc” refers to molecule or sequence comprising the sequence of a non-antigen-binding fragment resulting from digestion of whole antibody, whether in monomeric or multimeric form. The original immunoglobulin source of the native Fc is preferably of human origin and may be any of the immunoglobulins, although IgGI and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgAl, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “Fc variant” refers to a molecule or sequence that is modified from a native Fc but still comprises a binding site for the salvage receptor, FcRn. International applications WO 97/34631 (published Sep. 25, 1997) and WO 96/32478 describe exemplary Fc variants, as well as interaction with the salvage receptor, and are hereby incorporated by reference in their entirety. Thus, the term “Fc variant” comprises a molecule or sequence that is humanized from a non-human native Fc. Furthermore, a native Fc comprises sites that may be removed because 5 they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal lo heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by other means.

The term “multimer” as applied to Fc domains or molecules comprising Fc domains refers to molecules having two or more polypeptide chains associated covalently, noncovalently, or by both covalent and non-covalent interactions. IgG molecules typically form dimers; IgM, pentamers; IgD, dimers; and IgA, monomers, dimers, trimers, or tetramers. Multimers may be formed by exploiting the sequence and resulting activity of the native Ig source of the Fc or by derivatizing (as defined below) such a native Fc.

The term “dimer” as applied to Fc domains or molecules comprising Fc domains refers to molecules having two polypeptide chains associated covalently or non-covalently. Thus, exemplary dimers within the scope of this invention are as shown in FIG. 2.

The terms “derivatizing” and “derivative” or “derivatized” comprise processes and resulting compounds respectively in which (1) the compound has a cyclic portion; for example, cross-linking between cysteinyl residues within the compound; (2) the compound is cross-linked or has a cross-linking site; for example, the compound has a cysteinyl residue and thus forms cross-linked dimers in culture or in vivo; (3) one or more peptidyl linkage is replaced by a non-peptidyl linkage; (4) the N-terminus is replaced by —NRR¹, NRC(O)R¹, —NRC(O)OR¹, —NRS(O)₂R¹, —NHC(O)NHR, a succinimide group, or substituted or unsubstituted benzyloxycarbonyl-NH—, wherein R and R¹ and the ring substituents are as defined hereinafter; (5) the C-terminus is replaced by —C(O)R² or —NR³R⁴ wherein R², R³ and R⁴ are as defined hereinafter; and (6) compounds in which individual amino acid moieties are modified through treatment with agents capable of reacting with selected side chains or terminal residues. Derivatives are further described hereinafter.

The term “peptide” refers to molecules of 2 to 90 amino acids, with molecules of 5 to 85 amino acids preferred. Exemplary peptides may comprise the CT receptor modulating domain of a naturally occurring molecule or comprise randomized sequences.

The term “randomized” as used to refer to peptide sequences refers to fully random sequences (e.g., selected by phage display methods or RNA-peptide screening) and sequences in which one or more residues of a naturally occurring molecule is replaced by an amino acid residue not appearing in that position in the naturally occurring molecule. Exemplary methods for identifying peptide sequences include yeast-based screening, phage display, E. coli display, ribosome display, RNA-peptide screening, chemical screening, rational design, protein structural analysis, and the like.

The term “CT receptor modulating domain” refers to any amino acid sequence that binds to the CT receptor and comprises naturally occurring sequences or randomized sequences. Exemplary CT receptor modulating domains can be identified or derived as described in the references appearing in Table 1, which are hereby incorporated by reference in their entirety.

The term “CT receptor agonist” refers to a molecule that binds to the CT receptor and increases or decreases one or more CT receptor activity assay parameters as does its native ligand. An exemplary CT receptor activity assay is disclosed in Example 2.

The term “CT receptor antagonist” refers to a molecule that binds to the CT receptor and increases or decreases one or more assay parameters opposite from the effect on those parameters by CT. An exemplary CT receptor activity assay is disclosed in Example 2.

Additionally, physiologically acceptable salts of the compounds of this invention are also encompassed herein. The term “physiologically acceptable salts” refers to any salts that are known or later discovered to be pharmaceutically acceptable. Some specific examples are: acetate; trifluoroacetate; hydrohalides, such as hydrochloride and hydrobromide; sulfate; citrate; tartrate; glycolate; and oxalate.

Structure of compounds

In General.

CT receptor binding amino acid sequences are described in Table 1. Other information on CT receptor is found in Lenz et al. (1988), Regul. Peptides 12:351-357; Cardona, J. M. (1997), Osteoporosis Int. 7(3):165-174; Gennari et al. (1985), Curr. Ther. Res. 38(2):298-308. Each of these references is hereby incorporated by reference in its entirety.

The present inventors identified particular preferred known or naturally occurring sequences. These sequences can be randomized through the techniques mentioned above by which one or more amino acids may be changed while maintaining or even improving the binding affinity of the peptide.

In the compositions of matter prepared in accordance with this invention, the peptide may be attached to the vehicle through the peptide's N-terminus or C-terminus. Thus, the vehicle-peptide molecules of this invention may be described by the following formula I:

(A¹)ₐ-F¹-(A²)_{b} I

wherein:

F¹ is a vehicle (preferably an Fc domain);

A¹ and A² are each independently selected from -(L¹)_{c}-P¹, -(L¹)_{c}-P¹-(L²)_{d}-P², -(L¹)_{c}-P¹-(L²)_{d}-P²-(L³)ₑ-P³, and -(L¹)_{c}-P¹-(L²)_{d}-P²-(L³)ₑ-P³-(L⁴)_{f}-P⁴

P¹, P², P³, and P⁴ are each independently sequences of CT receptor modulating domains;

L¹, L², L³, and L⁴ are each independently linkers; and

a, b, c, d, e, and f are each independently 0 or 1, provided that at least one of a and b is 1.

Thus, compound I comprises preferred compounds of the formulae

A¹-F¹ II

and multimers thereof wherein F¹ is an Fc domain and is attached at the C-terminus of A¹;

F¹-A² III

and multimers thereof wherein F¹ is an Fc domain and is attached at the N-terminus of A²;

F¹-(L¹)_{c}-P¹ IV

and multimers thereof wherein F¹ is an Fc domain and is attached at the N-terminus of -(L¹)_{c}-P¹; and

F¹-(L¹)_{c}-P¹-(L²)_{d}-P² V

and multimers thereof wherein F¹ is an Fc domain and is attached at the N-terminus of -L¹-P¹-L²-P².

Peptides.

Any number of peptides may be used in conjunction with the present invention. Peptides may comprise part of the sequence of naturally occurring proteins, may be randomized sequences derived from the sequence of the naturally occurring proteins, or may be wholly randomized sequences. Phage display and RNA-peptide screening, in particular, are useful in generating peptides for use in the present invention.

A CT receptor modulating domain sequence particularly of interest is of the formula

CX²X³LSTCX⁸LX¹⁰X¹¹X¹²X¹³X¹⁴X¹⁵X¹⁶X¹⁷X¹⁸X¹⁹X²⁰X²¹X²²X²³X²⁴X²⁵X²⁶X²⁷GX²⁹X³⁰X³¹P (SEQ ID NO: 7)

wherein:

X² is a nonfunctional or hydrophilic residue (A, G, or S preferred);

X³ is a hydrophilic residue (N or S preferred);

X⁸ is a nonfunctional residue (M or V preferred);

X¹⁰ is a nonfunctional or hydrophilic residue (G or S preferred);

X¹¹ is a nonfunctional, basic, or hydrophilic residue (A, K, or T preferred);

X¹² is a nonfunctional or aromatic residue (L or Y preferred);

X¹³ is a hydrophilic or aromatic residue (S, T, or W preferred);

X¹⁴ is a basic or hydrophilic residue (Q or R preferred);

X¹⁵ is an acidic or hydrophilic residue (D, E, or N preferred);

X¹⁶ is a nonfunctional or aromatic residue (F or L preferred);

X¹⁷ is a basic or hydrophilic residue (H, K, or N preferred);

X¹⁸ is a basic or hydrophilic residue (K or N preferred);

X¹⁹ is an aromatic or nonfunctional residue (F or L preferred);

X²⁰ is a basic or hydrophilic residue (H or Q preferred);

X²¹ is a basic or hydrophilic residue (R or T preferred);

X²² is an aromatic residue (F or Y preferred);

X²³ is prolyl or a nonfunctional, hydrophilic, or aromatic residue (P or S preferred);

X²⁴ is a nonfunctional, hydrophilic or basic residue (G, Q, or R preferred);

X²⁵ is a nonfunctional or hydrophilic residue (M or T preferred);

X²⁶ is a nonfunctional, hydrophilic, or acidic residue (A, D, G, N, or S preferred);

X²⁷ is an aromatic, nonfunctional, or hydrophilic residue (F, I, T, or V preferred);

X²⁹ is prolyl or a nonfunctional, aromatic, or hydrophilic residue (A, P, S, or V preferred;

X³⁰ is an acidic or nonfunctional residue (E or G preferred); and

X³¹ is a nonfunctional or hydrophilic residue (A or T preferred).

Exemplary peptide sequences for this invention appear in Table 1 below. Molecules of this invention incorporating these peptide sequences may be prepared by methods known in the art. Single letter amino acid abbreviations are used. Any of these peptides may be linked in tandem (i.e., sequentially), with or without linkers, and a few tandem-linked examples are provided in the table. Any peptide containing a cysteinyl residue may be cross-linked with another Cys-containing peptide, either or both of which may be linked to a vehicle. Any peptide having more than one Cys residue may form an intrapeptide disulfide bond, as well. Any of these peptides may be derivatized as described hereinafter.

TABLE 1
CT Receptor modulating domains

| | | SEQ |
|---|---|---|
| Description | Sequence | ID NO: |
| salmon CT | SVSEIQLMHNLGKHL | 8 |
| | NSMERVEWLRKKLQ | |
| | DVHNFVALGAPLAPR | |
| | DAGSQRPRKKEDNV | |
| | LVESHEKSLGEADKA | |
| | DVNVLTKAKSQ | |
| human CT | CGNLSTCMLGTYTQ | 9 |
| | DFNKFHTFPQTAIGV | |
| | GAP | |
| eel CT | CSNLSTCVLGKLSQE | 10 |
| | LHKLQTYPRTDVGAG | |
| | TP | |
| porcine CT | CSNLSTCFLSAYWRN | 11 |
| | LNNFHRFSGMGFGP | |
| | ETP | |
| chicken CT | CASLSTCVLGKLSQE | 12 |
| | LHKLQTYPRTDVGAG | |
| | TP | |
| rat CT | CGNLSTCMLGTYTQ | 13 |
| | DLNKFHTFPQTSIGV | |
| | GAP | |
| human | ACDTATCVTHRLAGL | 14 |
| α-CGRP | LSRSGGVVKNNFVPT | |
| | NVGSKAF | |

Additional useful peptide sequences may result from conservative and/or non-conservative modifications of the amino acid sequences of SEQ ID NO: 7 or of those sequences appearing in Table 1.

Conservative modifications will produce peptides having functional and chemical characteristics similar to those of the peptide from which such modifications are made. In contrast, substantial modifications in the functional and/or chemical characteristics of the peptides may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the CT receptor site, or (c) the size of the molecule.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., 1998, Acta Physiol. Scand. Suppl. 643:55-67; Sasaki et al., 1998, Adv. Biophys. 35:1-24, which discuss alanine scanning mutagenesis).

Desired amino acid substitutions (whether conservative or non-conservative) can be determined by those skilled in the art at the time such substitutions are desired. For example, amino acid substitutions can be used to identify important residues of the peptide sequence, or to increase or decrease the affinity of the peptide or vehicle-peptide molecules (see preceding formulae) described herein. Exemplary amino acid substitutions are set forth in Table 2.

TABLE 2
Amino Acid Substitutions

| Original | Exemplary | Preferred |
|---|---|---|
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, | Leu |
| | Phe, Norleucine | |
| Leu (L) | Norleucine, Ile, Val, | Ile |
| | Met, Ala, Phe | |
| Lys (K) | Arg, 1,4 Diamino- | Arg |
| | butyric Acid, Gln, Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, | Leu |
| | Ala, Norleucine | |

In certain embodiments, conservative amino acid substitutions also encompass non-naturally occurring amino acid residues which are typically incorporated by chemical peptide synthesis rather than by synthesis in biological systems.

As noted in the foregoing section “Definition of Terms,” naturally occurring residues may be divided into classes based on common sidechain properties that may be useful for modifications of sequence. For example, non-conservative substitutions may involve the exchange of a member of one of these classes for a member from another class. Such substituted residues may be introduced into regions of the peptide that are homologous with non-human orthologs, or into the non-homologous regions of the molecule. In addition, one may also make modifications using P or G for the purpose of influencing chain orientation.

In making such modifications, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157: 105-131 (1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred. One may also identify epitopes from primary amino acid sequences on the basis of hydrophilicity. These regions are also referred to as “epitopic core regions.” A skilled artisan will be able to determine suitable variants of the polypeptide as set forth in the foregoing sequences using well known techniques. For identifying suitable areas of the molecule that may be changed without destroying activity, one skilled in the art may CT receptor areas not believed to be important for activity. For example, when similar polypeptides with similar activities from the same species or from other species are known, one skilled in the art may compare the amino acid sequence of a peptide to similar peptides. With such a comparison, one can identify residues and portions of the molecules that are conserved among similar polypeptides. It will be appreciated that changes in areas of a peptide that are not conserved relative to such similar peptides would be less likely to adversely affect the biological activity and/or structure of the peptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the peptide structure.

Additionally, one skilled in the art can review structure-function studies identifying residues in similar peptides that are important for activity or structure. In view of such a comparison, one can predict the importance of amino acid residues in a peptide that correspond to amino acid residues that are important for activity or structure in similar peptides. One skilled in the art may opt for chemically similar amino acid substitutions for such predicted important amino acid residues of the peptides.

One skilled in the art can also analyze the three-dimensional structure and amino acid sequence in relation to that structure in similar polypeptides. In view of that information, one skilled in the art may predict the alignment of amino acid residues of a peptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays know to those skilled in the art. Such data could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult J., Curr. Op. in Biotech., 7(4): 422-427 (1996), Chou et al., Biochemistry, 13(2): 222-245 (1974); Chou et al., Biochemistry, 113(2): 211-222 (1974); Chou et al., Adv. Enzymol. Relat. Areas Mol. Biol., 47: 45-148 (1978); Chou et al., Ann. Rev. Biochem., 47: 251-276 and Chou et al., Biophys. J., 26: 367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure. See Holm et al., Nucl. Acid. Res., 27(1): 244-247 (1999). It has been suggested (Brenner et al., Curr. Op. Struct. Biol., 7(3): 369-376 (1997)) that there are a limited number of folds in a given polypeptide or protein and that once a critical number of structures have been resolved, structural prediction will gain dramatically in accuracy.

Additional methods of predicting secondary structure include “threading” (Jones, D., Curr. Opin. Struct. Biol., 7(3): 377-87 (1997); Sippl et al.,Structure, 4(1): 15-9 (1996)), “profile analysis” (Bowie et al., Science, 253: 164-170 (1991); Gribskov et al., Meth. Enzym., 183: 146-159 (1990); Gribskov et al., Proc. Nat. Acad. Sci., 84(13): 4355-8 (1987)), and “evolutionary linkage” (See Home, supra, and Brenner, supra).

Vehicles.

This invention requires the presence of at least one vehicle (F¹, F²) attached to a peptide through the N-terminus, C-terminus or a sidechain of one of the amino acid residues. Multiple vehicles may also be used; e.g., Fc's at each terminus or an Fc at a terminus and a PEG group at the other terminus or a sidechain.

An Fc domain is the preferred vehicle. The Fc domain may be fused to the N or C termini of the peptides or at both the N and C termini. For the TPO-mimetic peptides, molecules having the Fc domain fused to the N terminus of the peptide portion of the molecule are more bioactive than other such fusions, so fusion to the N terminus is preferred.

As noted above, Fc variants are suitable vehicles within the scope of this invention. A native Fc may be extensively modified to form an Fc variant in accordance with this invention, provided binding to the salvage receptor is maintained; see, for example WO 97/34631 and WO 96/32478. In such Fc variants, one may remove one or more sites of a native Fc that provide structural features or functional activity not required by the fusion molecules of this invention. One may remove these sites by, for example, substituting or deleting residues, inserting residues into the site, or truncating portions containing the site. The inserted or substituted residues may also be altered amino acids, such as peptidomimetics or D-amino acids. Fc variants may be desirable for a number of reasons, several of which are described below. Exemplary Fc variants include molecules and sequences in which:

1. Sites involved in disulfide bond formation are removed. Such removal may avoid reaction with other cysteine-containing proteins present in the host cell used to produce the molecules of the invention. For this purpose, the cysteine-containing segment at the N-terminus may be truncated or cysteine residues may be deleted or substituted with other amino acids (e.g., alanyl, seryl). In particular, one may truncate the N-terminal 20-amino acid segment of SEQ ID NO: 2 or delete or substitute the cysteine residues at positions 7 and 10 of SEQ ID NO: 2. Even when cysteine residues are removed, the single chain Fc domains can still form a dimeric Fc domain that is held together non-covalently.

2. A native Fc is modified to make it more compatible with a selected host cell. For example, one may remove the PA sequence near the N-terminus of a typical native Fc, which may be recognized by a digestive enzyme in E. coli such as proline iminopeptidase. One may also add an N-terminal methionine residue, especially when the molecule is expressed recombinantly in a bacterial cell such as E. coli. The Fc domain of SEQ ID NO: 2 is one such Fc variant.

3. A portion of the N-terminus of a native Fc is removed to prevent N-terminal heterogeneity when expressed in a selected host cell. For this purpose, one may delete any of the first 20 amino acid residues at the N-terminus, particularly those at positions 1, 2, 3, 4 and 5.

4. One or more glycosylation sites are removed. Residues that are typically glycosylated (e.g., asparagine) may confer cytolytic response. Such residues may be deleted or substituted with unglycosylated residues (e.g., alanine).

5. Sites involved in interaction with complement, such as the C1q binding site, are removed. For example, one may delete or substitute the EKK sequence of human IgG1. Complement recruitment may not be advantageous for the molecules of this invention and so may be avoided with such an Fc variant.

6. Sites are removed that affect binding to Fc receptors other than a salvage receptor. A native Fc may have sites for interaction with certain white blood cells that are not required for the fusion molecules of the present invention and so may be removed.

7. The ADCC site is removed. ADCC sites are known in the art; see, for example, Molec. Immunol. 29 (5): 633-9 (1992) with regard to ADCC sites in IgG1. These sites, as well, are not required for the fusion molecules of the present invention and so may be removed.

8. When the native Fc is derived from a non-human antibody, the native Fc may be humanized. Typically, to humanize a native Fc, one will substitute selected residues in the non-human native Fc with residues that are normally found in human native Fc. Techniques for antibody humanization are well known in the art.

Preferred Fc variants include the following. In SEQ ID NO: 2 (FIG. 4) the leucine at position 15 may be substituted with glutamate; the glutamate at position 99, with alanine; and the lysines at positions 101 and 103, with alanines. In addition, one or more tyrosine residues can be replaced by phenyalanine residues.

An alternative vehicle would be a protein, polypeptide, peptide, antibody, antibody fragment, , or small molecule (e.g., a peptidomimetic compound) capable of binding to a salvage receptor. For example, one could use as a vehicle a polypeptide as described in U.S. Pat. No. 5,739,277, issued Apr. 14, 1998 to Presta et al. Peptides could also be selected by phage display or RNA-peptide screening or other techniques mentioned herein for binding to the FcRn salvage receptor. Such salvage receptor-binding compounds are also included within the meaning of “vehicle” and are within the scope of this invention. Such vehicles should be selected for increased half-life (e.g., by avoiding sequences recognized by proteases) and decreased immunogenicity (e.g., by favoring non-immunogenic sequences, as discovered in antibody humanization).

As noted above, polymer vehicles may also be used for F¹ and F² Various means for attaching chemical moieties useful as vehicles are currently available, see, e.g., Patent Cooperation Treaty (“PCT”) International Publication No. WO 96/11953, entitled “N-Terminally Chemically Modified Protein Compositions and Methods,” herein incorporated by reference in its entirety. This PCT publication discloses, among other things, the selective attachment of water soluble polymers to the N-terminus of proteins.

A preferred polymer vehicle is polyethylene glycol (PEG). The PEG group may be of any convenient molecular weight and may be linear or branched. The average molecular weight of the PEG will preferably range from about 2 kiloDalton (“kD”) to about 100 kD, more preferably from about 5 kD to about 50 kD, most preferably from about 5 kD to about 10 kD. The PEG groups will generally be attached to the compounds of the invention via acylation or reductive alkylation through a reactive group on the PEG moiety (e.g., an aldehyde, amino, thiol, or ester group) to a reactive group on the inventive compound (e.g., an aldehyde, amino, or ester group).

A useful strategy for the PEGylation of synthetic peptides consists of combining, through forming a conjugate linkage in solution, a peptide and a PEG moiety, each bearing a special functionality that is mutually reactive toward the other. The peptides can be easily prepared with conventional solid phase synthesis (see, for example, FIGS. 5 and 6 and the accompanying text herein). The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Polysaccharide polymers are another type of water soluble polymer which may be used for protein modification. Dextrans are polysaccharide polymers comprised of individual subunits of glucose predominantly linked by α1-6 linkages. The dextran itself is available in many molecular weight ranges, and is readily available in molecular weights from about 1 kD to about 70 kD. Dextran is a suitable water soluble polymer for use in the present invention as a vehicle by itself or in combination with another vehicle (e.g., Fc). See, for example, WO 96/11953 and WO 96/05309. The use of dextran conjugated to therapeutic or diagnostic immunoglobulins has been reported; see, for example, European Patent Publication No. 0 315 456, which is hereby incorporated by reference in its entirety. Dextran of about 1 kD to about 20 kD is preferred when dextran is used as a vehicle in accordance with the present invention.

Linkers.

Any “linker” group is optional. When present, its chemical structure is not critical, since it serves primarily as a spacer. The linker is preferably made up of amino acids linked together by peptide bonds. Thus, in preferred embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines. Other specific examples of linkers are:

(Gly)₃Lys(Gly)₄ (SEQ ID NO: 3);

(Gly)₃AsnGlySer(Gly)₂ (SEQ ID NO: 4);

(Gly)₃Cys(Gly)₄ (SEQ ID NO: 5); and

GlyProAsnGlyGly (SEQ ID NO: 6).

To explain the above nomenclature, for example, (Gly)₃Lys(Gly)₄ means Gly-Gly-Gly-Lys-Gly-Gly-Gly-Gly (SEQ ID NO: 3). Combinations of Gly and Ala are also preferred. The linkers shown here are exemplary; linkers within the scope of this invention may be much longer and may include other residues.

Non-peptide linkers are also possible. For example, alkyl linkers such as —NH—(CH₂)ₛ—C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker,

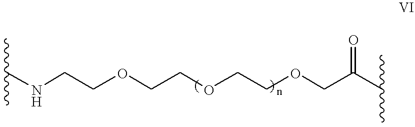

wherein n is such that the linker has a molecular weight of 100 to 5000 kD, preferably 100 to 500 kD. The peptide linkers may be altered to form derivatives in the same manner as described above.

Derivatives.

The inventors also contemplate derivatizing the peptide and/or vehicle portion of the compounds. Such derivatives may improve the solubility, absorption, biological half life, and the like of the compounds. The moieties may alternatively eliminate or attenuate any undesirable side-effect of the compounds and the like. Exemplary derivatives include compounds in which:

1. The compound or some portion thereof is cyclic. For example, the peptide portion may be modified to contain two or more Cys residues (e.g., in the linker), which could cyclize by disulfide bond formation.

2. The compound is cross-linked or is rendered capable of cross-linking between molecules. For example, the peptide portion may be modified to contain one Cys residue and thereby be able to form an intermolecular disulfide bond with a like molecule. The compound may also be cross-linked through its C-terminus, as in the molecule shown below.

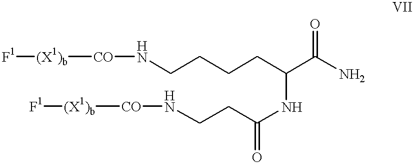

3. One or more peptidyl [—C(O)NR—] linkages (bonds) is replaced by a non-peptidyl linkage. Exemplary non-peptidyl linkages are —CH₂-carbamate [—CH₂—OC(O)NR—], phosphonate, —CH₂-sulfonamide [—CH₂—S(O)₂NR—], urea [—NHC(O)NH—], —CH₂-secondary amine, and alkylated peptide [—C(O)NR⁶— wherein R⁶ is lower alkyl].

4. The N-terminus is derivatized. Typically, the N-terminus may be acylated or modified to a substituted amine. Exemplary N-terminal derivative groups include —NRR¹ (other than —NH₂), —NRC(O)R¹, —NRC(O)OR¹, —NRS(O)₂R¹, —NHC(O)NHR¹, succinimide, or benzyloxycarbonyl-NH— (CBZ—NH—), wherein R and R¹ are each independently hydrogen or lower alkyl and wherein the phenyl ring may be substituted with 1 to 3 substituents selected from the group consisting of C₁-C₄ alkyl, C₁-C₄ alkoxy, chloro, and bromo.

5. The free C-terminus is derivatized. Typically, the C-terminus is esterified or amidated. Exemplary C-terminal derivative groups include, for example, —C(O)R² wherein R² is lower alkoxy or —NR³R⁴ wherein R³ and R⁴ are independently hydrogen or C₁-C₈ alkyl (preferably C₁-C₄ alkyl).

6. A disulfide bond is replaced with another, preferably more stable, cross-linking moiety (e.g., an alkylene). See, e.g., Bhatnagar et al. (1996), J. Med. Chem. 39: 3814-9; Alberts et al. (1993) Thirteenth Am. Pep. Symp., 357-9.

7. One or more individual amino acid residues is modified. Various derivatizing agents are known to react specifically with selected sidechains or terminal residues, as described in detail below.

Lysinyl residues and amino terminal residues may be reacted with succinic or other carboxylic acid anhydrides, which reverse the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with any one or combination of several conventional reagents, including phenylglyoxal, 2,3-butanedione, 1,2-cyclohexanedione, and ninhydrin. Derivatization of arginyl residues requires that the reaction be performed in alkaline conditions because of the high pKa of the guanidine functional group. Furthermore, these reagents may react with the groups of lysine as well as the arginine epsilon-amino group.

Specific modification of tyrosyl residues has been studied extensively, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl sidechain groups (aspartyl or glutamyl) may be selectively modified by reaction with carbodiimides (R′—N═C═N—R) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions.

Glutaminyl and asparaginyl residues may be deamidated to the corresponding glutamyl and aspartyl residues. Alternatively, these residues are deamidated under mildly acidic conditions. Either form of these residues falls within the scope of this invention.

Cysteinyl residues can be replaced by amino acid residues or other moieties either to eliminate disulfide bonding or, conversely, to stabilize cross-linking. See, e.g., Bhatnagar et al. (1996), J. Med. Chem. 39: 3814-9.

Derivatization with bifunctional agents is useful for cross-linking the peptides or their functional derivatives to a water-insoluble support matrix or to other macromolecular vehicles. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W. H. Freeman & Co., San Francisco), pp. 79-86 (1983).

Compounds of the present invention may be changed at the DNA level, as well. The DNA sequence of any portion of the compound may be changed to codons more compatible with the chosen host cell. For E. coli, which is the preferred host cell, optimized codons are known in the art. Codons may be substituted to eliminate restriction sites or to include silent restriction sites, which may aid in processing of the DNA in the selected host cell. The vehicle, linker and peptide DNA sequences may be modified to include any of the foregoing sequence changes.

Methods of Making

The compounds of this invention largely may be made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the peptides in an appropriate host. The vector comprises the DNA molecule that codes for the peptides operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides.

Compounds that contain derivatized peptides or which contain non-peptide groups may be synthesized by well-known organic chemistry techniques.

Uses of the Compounds

The compounds of this invention have pharmacologic activity resulting from their interaction with the CT receptor.

Agonists of the CT receptor are useful in treating:

various forms of osteoporosis, such as:

primary osteoporosis;

post-menopausal and age-related osteoporosis;

endocrine osteoporosis (hyperthyroidism, hyperparathyroidism, Cushing's syndrome, and acromegaly);

hereditary and congenital forms of osteoporosis (e.g., osteogenesis imperfecta, homocystinuria, Menkes' syndrome, and Riley-Day syndrome);

osteoporosis due to immobilization of extremities;

osteoporosis secondary to other disorders, such as hemochromatosis, hyperprolactinemia, anorexia nervosa, thyrotoxicosis, diabetes mellitus, celiac disease, inflammatory bowel disease, primary biliary cirrhosis, rheumatoid arthritis, ankylosing spondylitis, multiple myeloma, lymphoproliferative diseases, and systemic mastocytosis;

osteoporosis secondary to surgery (e.g., gastrectomy) or to drug therapy, such as chemotherapy, anticonvulsant therapy, immunosuppressive therapy, and anticoagulant therapy;

pain, particularly pain related to cancer;

and the like.

Pharmaceutical Compositions

In General.

The present invention also provides methods of using pharmaceutical compositions of the inventive compounds. Such pharmaceutical compositions may be for administration for injection, or for oral, pulmonary, nasal, transdermal or other forms of administration. In general, the invention encompasses pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference in their entirety. The compositions may be prepared in liquid form, or may be in dried powder, such as lyophilized form. Implantable sustained release formulations are also contemplated, as are transdermal formulations.

Oral Dosage Forms.

Contemplated for use herein are oral solid dosage forms, which are described generally in Chapter 89 of Remington's Pharmaceutical Sciences (1990), 18th Ed., Mack Publishing Co. Easton Pa. 18042, which is herein incorporated by reference in its entirety. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given in Chapter 10 of Marshall, K., Modern Pharmaceutics (1979), edited by G. S. Banker and C. T. Rhodes, herein incorporated by reference in its entirety. In general, the formulation will include the inventive compound, and inert ingredients which allow for protection against the stomach environment, and release of the biologically active material in the intestine.

Also specifically contemplated are oral dosage forms of the above inventive compounds. If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Moieties useful as covalently attached vehicles in this invention may also be used for this purpose. Examples of such moieties include: PEG, copolymers of ethylene glycol and propylene glycol, carboxymethyl cellulose, dextran, polyvinyl alcohol, polyvinyl pyrrolidone and polyproline. See, for example, Abuchowski and Davis, Soluble Polymer-Enzyme Adducts, Enzymes as Drugs (1981), Hocenberg and Roberts, eds., Wiley-Interscience, New York, NY,, pp. 367-83; Newmark, et al. (1982), J. Appl. Biochem. 4:185-9. Other polymers that could be used are poly-1,3-dioxolane and poly-1,3,6-tioxocane. Preferred for pharmaceutical usage, as indicated above, are PEG moieties.

For oral delivery dosage forms, it is also possible to use a salt of a modified aliphatic amino acid, such as sodium N-(8-[2-hydroxybenzoyl] amino) caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

The compounds of this invention can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the compound of the invention with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the compound of this invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives may also be included in the formulation to enhance uptake of the compound. Additives potentially having this property are for instance the fatty acids oleic acid, linoleic acid and linolenic acid.

Controlled release formulation may be desirable. The compound of this invention could be incorporated into an inert matrix which permits release by either diffusion or leaching mechanisms e.g., gums. Slowly degenerating matrices may also be incorporated into the formulation, e.g., alginates, polysaccharides. Another form of a controlled release of the compounds of this invention is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Pulmonary Delivery Forms.

Also contemplated herein is pulmonary delivery of the present protein (or derivatives thereof). The protein (or derivative) is delivered to the lungs of a mammal while inhaling and traverses across the lung epithelial lining to the blood stream. (Other reports of this include Adjei et al., Pharma. Res. (1990) 7: 565-9; Adjei et al. (1990), Internatl. T. Pharmaceutics 63: 135-44 (leuprolide acetate); Braquet et al. (1989), J. Cardiovasc. Pharmacol. 13 (suppl.5): s.143-146 (endothelin-1); Hubbard et al. (1989), Annals Int. Med. 3: 206-12 (α1-antitrypsin); Smith et al. (1989), J. Clin. Invest. 84: 1145-6 (α1-proteinase); Oswein et al. (March 1990), “Aerosolization of Proteins”, Proc. Symp. Resp. Drug Delivery II, Keystone, Colo. (recombinant human growth hormone); Debs et al. (1988), J. Immunol. 140: 3482-8 (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

The inventive compound should most advantageously be prepared in particulate form with an average particle size of less than 10 μm (or microns), most preferably 0.5 to 5 μm, for most effective delivery to the distal lung.

Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise the inventive compound dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the inventive compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent, such as lactose, sorbitol, sucrose, mannitol, trehalose, or xylitol in amounts which facilitate dispersal of the powder from the device, e.g., 50 to 90% by weight of the formulation.

Nasal Delivery Forms.

Nasal delivery of the inventive compound is also contemplated. Nasal delivery allows the passage of the protein to the blood stream directly after administering the therapeutic product to the nose, without the necessity for deposition of the product in the lung. Formulations for nasal delivery include those with dextran or cyclodextran. Delivery via transport across other mucous membranes is also contemplated.

Dosages.

The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.1-1000 micrograms of the inventive compound per kilogram of body weight, preferably 0.1-150 micrograms per kilogram.

Specific Preferred Embodiments

The inventors have determined preferred structures for the preferred peptides listed in Table 3 below. The symbol “Λ” may be any of the linkers described herein or may simply represent a normal peptide bond (i.e., so that no linker is present). Tandem repeats and linkers are shown separated by dashes for clarity.

TABLE 3
Preferred embodiments

| | Peptide | |
|---|---|---|
| | descrip- | SEQ ID |
| Sequence/structure | tion | NO: |
| CSNLSTCVLGKLSQELHKLQTYPRTNTGS | salmon | 15 |
| GTP-Λ-F¹ | CT | |
| F^{1-Λ-CSNLSTCVLGKLSQELHKLQTYPRTNTG} | salmon | 16 |
| SGTP | CT | |
| CSNLSTCVLGKLSQELHKLQTYPRTNTGSGTP-Λ- | salmon | 17 |
| CSNLSTCVLGKLSQELHKLQTYPRTNTGSG | CT | |
| TP-Λ-F¹ | | |
| F^{1-Λ-CSNLSTCVLGKLSQELHKLQTYPRTNTGS} | salmon | 18 |
| GTP-Λ-CSNLSTCVLGKLSQELHKLQTYPRTNTG | CT | |
| SGTP | | |
| CGNLSTCMLGTYTQDFNKFHTFPQTAIGVGAP-Λ- | human CT | 19 |
| F¹ | | |
| F^{1-Λ-} | human CT | 20 |
| CGNLSTCMLGTYTQDFNKFHTFPQTAIGVGAP | | |
| CGNLSTCMLGTYTQDFNKFHTFPQTAIGVGAP-Λ- | human CT | 21 |
| CGNLSTCMLGTYTQDFNKFHTFPQTAIGVGAP-Λ- | | |
| F¹ | | |
| F^{1-Λ-} | human CT | 22 |
| CGNLSTCMLGTYTQDFNKFHTFPQTAIGVGAP-Λ- | | |
| CGNLSTCMLGTYTQDFNKFHTFPQTAIGVGAP | | |
| ACDTATCVTHRLAGLLSRSGGVVKNNFVPTNVGS | human α- | 23 |
| KAF-Λ-F¹ | CGRP | |
| F^{1-Λ-} | human α- | 24 |
| ACDTATCVTHRLAGLLSRSGGVVKNNFVPTNVGS | CGRP | |
| KAF | | |
| ACDTATCVTHRLAGLLSRSGGVVKNNFVPTNVGS | human α- | 25 |
| KAF-Λ- | CGRP | |
| ACDTATCVTHRLAGLLSRSGGVVKNNFVPTNVGS | | |
| KAF-Λ-F¹ | | |
| F1-Λ- | human α- | 26 |
| ACDTATCVTHRLAGLLSRSGGVVKNNFVPTNVGS | CGRP | |
| KAF-Λ- | | |
| ACDTATCVTHRLAGLLSRSGGVVKNNFVPTNVGS | | |
| KAF | | |

“F¹” is an Fc domain as defined previously herein. In addition to those listed in Table 3, the inventors further contemplate heterodimers in which each strand of an Fc dimer is linked to a different peptide sequence; for example, wherein each Fc is linked to a different sequence selected from Table 1.

All of the compounds of this invention can be prepared by methods described in PCT appl. no. WO 99/25044, which is hereby incorporated by reference in its entirety.

The invention will now be further described by the following working examples, which are illustrative rather than limiting.

### EXAMPLE 1

### Preparation of Fc-peptide Constructs

An Fc-CT fusion protein with a C-terminal amide can be prepared using a specially engineered protein splicing system. Protein splicing, an analogous phenomenon to RNA splicing, is a recently discovered posttranslational modification process in which a nascent protein undergoes spontaneous rearrangement resulting in the excision of an internal sequence (intein) and concomitant ligation of the flanking fragments (N- and C-terminal exteins) (FIG. 4).

The autocatalytic activity is exerted by the intein. Several conserved residues are found in all protein splicing systems: a Cys (Ser or The) at the C-terminal side of the two junctions and an Asn at the last and a His at the penultimate position of the intein (FIG. 4). The detailed mechanism, as shown in FIG. 5, involves an N to S (in the case of Cys) acyl transfer at the first step to form a thioester, followed by a transesterification reaction which transfers the N-extein acyl to the side chain of the first Cys residue of the C-extein, a subsequent succinimide formation involving the Asn residue resulting in the rupture of the Asn-Cys bond, and a final S to N acyl transfer to form a new peptide bond between the N-extein and C-extein.

Exploitation of the protein splicing system by mutational manipulation has generated several self-cleavage, rather than intein splicing, systems useful for bioseparations. For example, when the last Asn residue of intein is changed to Ala, the thioester intermediates can still form but the intein can no longer be excised off. Since a thioester is chemically reactive, it can be cleaved by a nucleophile. These unique features have made such systems very useful in biotechnology. One of the applications is affinity protein purification, where a large fusion protein containing a protein of interest, an intein and an affinity tag is constructed for the single-step purification of the interesting protein. The Fc-CT protein amide can certainly be prepared by using such a process (FIG. 6).

Methods.

The synthetic calcitonin portion of the Fc-calcitonin gene was synthesized using the following protocol. Oligonucleotides were synthesized on an Applied Biosystems 394 DNA synthesizer (Foster City, Calif.) using standard procedures, 5′-phosphorylation was achieved using a chemical phosphorylation reagent (Glenn Research, Sterling, Va.). After aminolysis, oligomers were purified by high-resolution denaturing PAGE. Lyophilized products were dissolved in deionized water at a concentration of 10 μM, as determined by UV spectroscopy. Ten picomoles of each oligonucleotide was used in a 50 μl ligase chain reaction containing 20 mM Tris-HCl (pH 7.5), 20 mM KCl, 10 mM MgCl2, 0.1% NP-40, 0.1 mM ATP, 1 mM DTT and 10U of Pfu DNA ligase (Stratagene, La Jolla, Calif.). Twenty-five cycles were performed using a Perkin-Elmer Thermal Cycler 480 (Foster City, Calif.) at 94° C. for 45 seconds and 60° C. for 1 minute. The resulting assembly reaction was PCR amplified in a 100 μl reaction containing 10 mM Tris-HCl (pH 8.8), 10 mM KCl, 0.02% Tween 20, 1 mM MgCl2, 50 uM each DNTP, 2 ul of ligation reaction, 2 pMoles of each primer, and 3 units of Pwo DNA polymerase (Perkin Elmer). The reaction was cycled 25 times on a Perkin Elmer 9600 Thermal Cycler (94° C. for 30 sec, 65° C. for 45 sec and 72° C. for 45 sec.) PCR products were analyzed on a 1% Nusieve gel.

The Fc gene fragment was PCR amplified in a 100 μl reaction containing 10 mM Tris-HCl (pH 8.8), 10 mM KCl, 0.02% Tween 20, 1 mM MgCl2, 50 uM each dNTP, 2 ul of ligation reaction, 2 pMoles of each primer, and 3 units of Pwo DNA polymerase (Perkin Elmer). The reaction was cycled 25 times on a Perkin Elmer 9600 Thermal Cycler (94° C. for 30 sec, 65° C. for 45 sec and 72° C. for 45 sec.) The resultant products were gel purified by running samples on a 3% NuSieve gel, cutting samples of the correct size out of the gel and purified using a Promega Wizard PCR prep purification column. It is important to note that a proofreading polymerase is required for this step.

The Fc-calcitonin fusion was done in a 50 μl volume with 10 mM Tris-HCl pH 8.8, 10 mM KCl, 0.002% (v/v) Tween 20, 5 mM MgCl2, 4 mM dNTPs, 2 ul of the Fc PCR product and 2 ul of the salmon calcitonin PCR product and placed in a thermocycler. The mixture was heated to 95 C for 5 minutes. In a 50 ul reaction volume, 10 mM Tris-HCl pH 8.8, 10 mM KCl, 0.002% (v/v) Tween 20 and 6 units of UlTma DNA polymerase was added to the heated PCR mixture. The reaction was cycled 10 times on a Perkin Elmer 9600 Thermal Cycler (95° C. for 45 sec, 63.1° C. for 1.00 min and 72° C. for 2.00 min.) Note that there are no primers in this reaction.

A 50 μl reaction containing 10 mM Tris-HCl pH 8.8, 10 mM KCl, 0.002% (v/v) Tween 20, 5 mM MgCl2, 4 mM dNTPs, 0.01 ng of the Fc-calcitonin fusion, 200 pMoles of the respective upstream PCR primer and 200 pMoles of the respective downstream PCR primer. The reaction tube was placed in the thermocycler, and heated to 95° C. for 5 minutes. In a 50 μl reaction volume, 10 mM Tris-HCl pH 8.8, 10 mM KCl, 0.002% (v/v) Tween 20 and 6 units of UlTma DNA polymerase was added to the heated PCR mixture. The reaction was cycled 25 times on a Perkin Elmer 9600 Thermal Cycler (95° C. for 45 sec, 63.1° C. for 1.00 min and 72° C. for 2.00 min.) A TBE 1% SeaKem gel was used. The resulting PCR product was cut with Nde I and Kpn I restriction enzymes (BM) and cloned into the pTYB 1 plasmid (New England Biolabs). The resulting ligation was transformed into E. coli strain ER2566 and analyzed. Positive colonies were confirmed by sequencing.

1 liter of Terrrific Broth containing 100 μg/ml ampicillin was inoculated with a freshly grown colony. The culture was placed in a shaking inubator at 37 C until the O.D. reached 0.8. 0.5 mM of IPTG was added before the culture was transferred to a 15 C incubated shaker for the night. The cells were spun down at 5000× g for 10 minutes at 4 C. After discarding the supernatant, the cell pellet was resuspended in 50 ml ice-cold Cell Lysis Buffer (20 mM Tris-HCl pH 8.0, 500 mM NaCl, 1 mM EDTA, 0.1% Triton X-100). The cells were broken by sonication and the supernatant was clarified by centrifugation at 12,000× g for 30 minutes. The clarified extract was applied slowly to a chitin column. The column was washed with 10 bed volumes of 20 mM Tris-HCl, 1 M NaCl, and 1 mM EDTA. The target protein was released from the chitin by 3 bed volumes of 250 mM NH4HCO3 containing 25 mM MESA left on the column overnight. The target protein was then eluted off and collected.

The Fc-calcitonin target protein was analyzed on a 10-20% tricine gel and stained with a Colloidal Blue staining kit and by reverse phase HPLC as well (see data, FIGS. 8A and 8B).

The invention now being fully described, it will be apparent to one of ordinary skill in the art that many changes and modifications can be made thereto, without departing from the spirit and scope of the invention as set forth herein.

What is claimed is:

1. A composition of matter of the formula
(A¹)ₐ-F¹-(A²)_{b}
and multimers thereof, wherein:
F¹ is a vehicle;
A¹ and A² are each independently selected from -(L¹)_{c}-P¹, -(L¹)_{c}-P¹-(L²)_{d}-P², -(L¹)_{c}-P¹-(L²)_{d}-P²-(L³)ₑ-P³ and -(L¹)_{c}-P¹-(L²)_{d}-P²-(L³)ₑ-P³-(L⁴)_{f}-P⁴
P¹, P², P³, and P⁴ are each independently sequences of CT receptor modulating domains;
L¹, L², L³, and L⁴ are each independently linkers; and
a, b, c, d, e, and f are each independently 0 or 1, provided that at least one of a and b is 1.

2. The composition of matter of claim 1 of the formulae
A¹-F¹
or
F¹-A².

3. The composition of matter of claim 1 of the formula
F¹-(L¹)_{c}-P¹.

4. The composition of matter of claim 1 of the formula
F¹-(L¹)_{c}-P¹-(L²)_{d}-P².

5. The composition of matter of claim 1, wherein F¹ is an Fc domain.

6. The composition of matter of claim 1 wherein F¹ is an IgG Fc domain.

7. The composition of matter of claim 1 wherein F¹ is an IgGl Fc domain.

8. The composition of matter of claim 1 wherein F¹ comprises the sequence of SEQ ID NO: 2.

9. The composition of matter of claim 1 wherein the CT receptor modulating domain sequence is of the formula
CX²X³LSTCX⁸LX¹⁰X¹¹X¹²X¹³X¹⁴X¹⁵X¹⁶X¹⁷X¹⁸X¹⁹X²⁰X²¹X²²X²³X²⁴X²⁵X²⁶x²⁷GX²⁹X³⁰X³¹P (SEQ ID NO: 7)
wherein:
X² is a nonfunctional or hydrophilic residue;
X³ is a hydrophilic residue;
X⁸ is a nonfunctional residue;
X¹⁰ is a nonfunctional or hydrophilic residue;
X¹¹ is a nonfunctional, basic, or hydrophilic residue;
X¹² X is a nonfunctional or aromatic residue;
X¹³ is a hydrophilic or aromatic residue;
X¹⁴ is a basic or hydrophilic residue;
X¹⁵ is an acidic or hydrophilic residue;
X¹⁶ is a nonfunctional or aromatic residue;
X¹⁷ is a basic or hydrophilic residue;
X¹⁸ is a basic or hydrophilic residue;
X¹⁹ is an aromatic or nonfunctional residue;
X²⁰ is a basic or hydrophilic residue;
X²¹ is a basic or hydrophilic residue;
X²² is an aromatic residue;
X²³ is prolyl or a nonfunctional, hydrophilic, or aromatic residue;
X²⁴ is a nonfunctional, hydrophilic or basic residue;
X²⁵ is a nonfunctional or hydrophilic residue;
X²⁶ is a nonfunctional, hydrophilic, or acidic residue;
X²⁷ is an aromatic, nonfunctional, or hydrophilic residue;
X²⁹ is prolyl or a nonfunctional, aromatic, or hydrophilic residue;
X³⁰ is an acidic or nonfunctional residue; and
X³¹ is a nonfunctional or hydrophilic residue.

10. The composition of matter of claim 9 wherein:
X² is A, G, or S;
X³is N or S;
X⁸ is M or V;
X¹⁰ is G or S;
X¹¹ is A, K, or T;
X¹² is L or Y;
X¹³ is S, T, or W;
X¹⁴ is Q or R;
X¹⁵ is D, E, or N;
X¹⁶ is F or L;
X¹⁷ is H, K, or N;
X^{18 is K or N;}
X¹⁹ is F or L;
X²⁰ is H or Q;
X²¹ is R or T;
X²² is F or Y;
X²³ is P or S;
X²⁴ is G, Q, or R;
X²⁵ is M or T;
X²⁶ is A, D, G, N, or S;
X²⁷ is F, I, T, or V;
X²⁹ is A, P, S, or V;
X³⁰ is E or G; and
X³ is A or T.

11. The composition of matter of claim 9, wherein F¹ is an Fc domain.

12. The composition of matter of claim 9, wherein F¹ is an IgG Fc domain.

13. The composition of matter of claim 11, wherein F¹ is an IgG1 Fc domain.

14. The composition of matter of claim 1, wherein the CT receptor modulating sequence is selected from Table 1 (SEQ ID NOS: 8 to 14).

15. The composition of matter of claim 9, wherein the CT receptor modulating sequence is selected from Table 1 (SEQ ID NOS: 8 to 14).

16. The composition of matter of claim 5, having a sequence selected from Table 3 (SEQ ID NOS: 15 to 26).

17. A DNA encoding a composition of matter of claim 5.

18. A DNA encoding a composition of matter of claim 10.

19. An expression vector comprising the DNA of claim 16.

20. An expression vector comprising the DNA of claim 17.

21. A host cell comprising the expression vector of claim 18.

22. A host cell comprising the expression vector of claim 19.

23. The cell of claim 20, wherein the cell is an E. coli cell.

24. The cell of claim 21, wherein the cell is an E. coli cell.

25. A process for preparing a modulator of the CT receptor, which comprises:
a. selecting at least one peptide that binds to the CT receptor; and
b. preparing a pharmacologic agent comprising at least one Fc domain covalently linked to at least one amino acid sequence of the selected peptide or peptides.

26. The process of claim 25, wherein the peptide comprises SEQ ID NO: 7.

27. The process of claim 25, wherein the peptide is selected in a process comprising one or more techniques selected from yeast-based screening, rational design, protein structural analysis, screening of a phage display library, an E. coli display library, a ribosomal library, an RNA-peptide library, and a chemical peptide library.

28. The process of claim 25, wherein the preparation of the pharmacologic agent is carried out by:
a. preparing a gene construct comprising a nucleic acid sequence encoding the selected peptide and a nucleic acid sequence encoding an Fc domain; and
b. expressing the gene construct.

29. The process of claim 28, wherein the gene construct is expressed in an E. coli cell.

30. The process of claim 25, wherein the selection of the peptide is carried out by a process comprising:
c. preparing a gene construct comprising a nucleic acid sequence encoding a first selected peptide and a nucleic acid sequence encoding an Fc domain;
d. conducting a polymerase chain reaction using the gene construct and mutagenic primers, wherein
i) a first mutagenic primer comprises a nucleic acid sequence complementary to a sequence at or near the 5′ end of a coding strand of the gene construct, and
ii) a second mutagenic primer comprises a nucleic acid sequence complementary to the 3′ end of the noncoding strand of the gene construct.

31. The compound of claim 5, wherein the C-terminus is amidated.

32. The process of claim 25, further comprising amidating the C-terminus of the modulator.

33. A method of treating osteoporosis, which comprises administering a composition of matter of claim 1.
